(19) United States
(12) Patent Application Publication
(10) Pub. No.: US 2022/0215902 A1
(43) Pub. Date: Jul. 7, 2022

(54) ANALYSIS METHOD FOR DETERMINING HAPLOTYPES OF FILIAL GENERATION OBJECTS AND DEVICE

(71) Applicant: YIKON GENOMICS (SUZHOU) CO., LTD, Suzhou (CN)

(72) Inventors: Yangyun ZOU, Suzhou (CN); Yingying XIA, Suzhou (CN); Sijia LU, Suzhou (CN); Chunxu HU, Suzhou (CN)

(21) Appl. No.: 17/597,904

(22) PCT Filed: Jul. 29, 2020
(86) PCT No.: PCT/CN2020/105527

§ 371 Date: Jan. 28, 2022

(30) Foreign Application Priority Data
Jul. 30, 2019 (CN) 2019 10696622.5

(51) Int. Cl. G16B 30/10; G16B 20/20; G16H 70/60

(52) U.S. Cl. CPC G16B 30/10; G16H 70/60; G16B 20/20

(57) ABSTRACT

The invention provides an analysis method and a device for determining a haplotype of a descendant object. Particularly, the invention provides a data analysis method for determining a haplotype genetic flow, comprising the following steps: (a) providing data sets for the analysis, the data sets being data sets related to genome information; (b) performing molecular marker genotyping in the upstream and downstream regions of Y1 target sites in each of the data sets, thereby obtaining molecular marker genotyping data, wherein Y1 is a positive integer greater than or equal to 1; (c) constructing a binary genetic vector of (0, 1) for each molecular marker site upstream and downstream of each target site in each of the data sets; (d) determining a maximum likelihood estimation value L using a Hidden Markov model for each target site; (e) determining a haplotype genetic flow direction of the descendant object and the family members through a Viterbi dynamic programming algorithm.

### TECHNICAL FIELD

The invention relates to the field of biomedicine and molecular cell biology, particularly to an analysis method and a device for determining a haplotype of a descendant object.

### BACKGROUND

Determination of haplotypes is of great significance for the kinship identification, scientific research, and the like. In current practice, the strategy of polymorphic site linkage analysis is also often used in PGT-M and PGT-SR-Balanced assays to assist in inference of disease state. In the PGT-M assay, due to the characteristics of allele dropout and uneven whole-genome amplification in single-cell whole-genome amplification, direct pathogenic site detection would lead to a certain false-positive rate or false-negative rate. Therefore, at present, the linkage and crossover theory of genes is often used to make further inferences by comparing the haplotype of an embryo with the haplotype of a reference sample having a known disease-carrying status; whereas in the PGT-SR-Balanced assay, samples with normal CNVs (copy number variations) cannot be directly detected by certain technical means such as low-depth whole-genome sequencing (CNV-Seq), gene chips, etc. However, the analytical capability or accuracy of these methods is far from satisfactory.

Therefore, there is an urgent need in the art to develop an analysis method for an effective and accurate analysis of the haplotype of a descendant object.

### SUMMARY OF THE INVENTION

The purpose of the invention is to provide a method and a device for an effective and accurate analysis of the haplotype of a descendant object.

In a first aspect, the invention provides a data analysis method for determining a haplotype genetic flow comprising the following steps:

(a) Providing data sets for the analysis, wherein the data sets are related to genome information and comprise: a first data set derived from a descendant object, a second data set derived from the father of the descendant object and/or a third data set derived from the mother of the descendant object, and a reference data set C derived from at least one reference object; wherein the total number of the first, second, and third data sets and the reference data set C is s;
wherein the reference object is a genetically related relative other than the father and the mother of the descendant object; and
provided that:
(1) when both the second data set and the third data set are present, s is a positive integer greater than or equal to 4;
(2) when the second data set is present and the third data set is absent, s is a positive integer greater than or equal to 3, and the reference object is a genetically related relative other than the father and the mother of the descendant object and is genetically related to the father; and
(3) when the third data set is present and second data set is absent, s is a positive integer greater than or equal to 3, and the reference object is a genetically related relative other than the father and the mother of the descendant object and is genetically related to the mother;
   (b) Performing molecular marker genotyping in the upstream and downstream regions of Y1 target sites in each of the data sets, thereby obtaining molecular marker genotype data, wherein Y1 is a positive integer greater than or equal to 1;
   (c) For each of the molecular marker sites upstream and downstream of each target site in each of the data sets, constructing binary genetic vectors of (0, 1); n data sets constitute 2n vectors of Vᵢ, wherein i represents a site, and Vᵢ is a Hidden Markov Chain state; wherein n is s or s-j, and s is as defined above, and j is the number of the uppermost ancestral individuals without a parental generation (i.e., individuals without parents in the pedigree);
   (d) For each target site, determining a maximum likelihood estimation value L by Formula Q1 using a Hidden Markov model:

$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$

wherein:
m represents the number of molecular markers upstream and downstream of each target site;
P(V₁) represents a priori value of a genetic vector;
P(Vᵢ|Vᵢ₋₁) represents a transition probability of the haplotype status between two adjacent sites;
Gᵢ represents an observed value of a genotype at the ith site;
P(Gᵢ|Vᵢ) represents an emission probability of a haplotype status; and
(e) Estimating a maximum possible composition of V₁, V₂, . . . Vₘ by using a Viterbi dynamic programming algorithm, thus a haplotype genetic flow direction of the descendant object and family members is determined.

In a second aspect, the invention provides an analysis method for determining a haplotype of a descendant object, comprising the following steps:

(i) Providing s data sets for the analysis, wherein s is a positive integer greater than or equal to 4, wherein the data sets are related to genome information and comprise: a first data set derived from the descendant object, a second data set derived from the father of the descendant object, a third data set derived from the mother of the descendant object, and at least one reference data set C from a reference object;
wherein the reference object is a genetically related relative other than the father and the mother of the descendant object;
(ii) Selecting Y1 target sites, wherein Y1 is a positive integer greater than or equal to 1;
(iii) For each target site selected out in the previous step, analyzing and detecting molecular markers in the upstream and downstream regions of the target site, so as to determine at least one molecular marker upstream of and at least one molecular marker downstream of each target site;
(iv) Annotating each of the molecular markers determined in step (iii) in each of the data sets to obtain the corresponding first data set, second data set, third data set and reference data set C annotated with the molecular markers;
(v) For each of the molecular marker sites upstream and downstream of each target site in each of the data sets, constructing binary genetic vectors of (0, 1); n data sets constitute 2n vectors of Vᵢ, wherein i represents a site, and Vᵢ is a Hidden Markov Chain state; wherein n is s or s-j, and s is as defined above, and j is the number of the uppermost ancestral individuals without a parental generation (i.e., individuals without parents in the pedigree);
(vi) For each target site, determining a maximum likelihood estimation value L by Formula Q1 using a Hidden Markov model:

$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$

wherein:
m represents the number of molecular markers upstream and downstream of each target site;
P(V₁) represents a priori value of a genetic vector;
P(Vᵢ|Vᵢ₋₁) represents a transition probability of the haplotype status between two adjacent sites;
Gᵢ represents an observed value of a genotype at the ith site;
P(Gᵢ|Vᵢ) represents an emission probability of a haplotype status; and
(vii) Estimating a maximum possible composition of V₁, V₂, . . . Vₘ by using a Viterbi dynamic programming algorithm, thus a haplotype of the descendant object is determined.

In another preferred embodiment, step (vii) comprises determining the abnormal mutation carrying status of the haplotype of the descendant object according to the genetic flow of the haplotype of the descendant object.

According to the method of the first aspect or the second aspect of the present invention, the P(Vᵢ|Vᵢ₋₁) is calculated by using a genetic map and obtaining a recombination rate; and/or the P(Gᵢ|Vᵢ) is a probability calculated by combining an observed value of a sample genotype and a genotype of an ancestor thereof, and using the Mendelian inheritance law.

In another preferred embodiment, Y1 is from 1 to 1000000, preferably from 100 to 500000, and more preferably from 1000 to 100000.

In another preferred embodiment, steps (v) to (vii) or steps (c) to (e) are performed simultaneously or sequentially for a plurality of target sites.

In another preferred embodiment, the target site is an abnormal mutation site or region.

In another preferred embodiment, the data sets related to genome information are data sets of genome DNA information.

In another preferred embodiment, step (e) further comprises determining a family inheritance pedigree of the descendant object according to the haplotype genetic flow.

In another preferred embodiment, the descendant object comprises animals.

In another preferred embodiment, the method is non-diagnostic and non-therapeutic.

In another preferred embodiment, the method is used to determine the genetic relationship between the descendant object and the father and mother of the descendant object.

According to the method of the first aspect or the second aspect of the present invention, the descendant object is selected from the group consisting of humans or non-human mammals.

According to the method of the first aspect or the second aspect of the present invention, the method further comprises one or more features selected from the group consisting of:

(1) The data set is consisting of sequencing data or chip detection data of genome nucleic acids;
(2) The upstream and downstream regions comprise: ≤1 Mbp region, ≤2 Mbp region, ≤3 Mbp region or up to an entire chromosome;
(3) the molecular marker is selected from the group consisting of a SNP site, a STR polymorphic site, a RFLP site, an AFLP site, or a combination thereof;
(4) The molecular marker detection means include a microarray chip of single nucleotide polymorphic sites, a MassARRAY flight mass spectrometry chip, a MLPA multiplex ligation amplification technique, a second-generation sequencing, a third-generation sequencing, or a combination thereof;
(5) The molecular marker detection identifies for each target abnormal mutation at least two molecular markers that may be linked, and are recorded as analysis sites.

In another preferred embodiment, each of the analysis sites in each of the data sets is annotated to obtain corresponding first data set, second data set, third data set, and the reference data set C with annotated analysis sites.

In another preferred embodiment, when the molecular marker is a SNP site, it can be that all sites in the preferred embodiment are heterozygous for the carrier and homozygous for the partner thereof in the SNP genotyping data.

In another preferred embodiment, step (iii) further comprises performing corresponding quality control on the molecular marker genotype data, so as to remove analysis sites that do not meet the quality control standards.

In another preferred embodiment, the quality control is selected from the group consisting of: quality control for single-cell whole-genome amplification efficiency, quality control for identification of Mendelian inheritance violation, quality control for identification of chromosomal interference theory (the phenomenon of mutual impact and suppression between two adjacent single crossover of non-sister chromatids during meiosis, suppression theory called positive crossover interference being used here), or a combination thereof.

In another preferred embodiment, the haplotype of abnormal mutation carrying status is an abnormal haplotype associated with a disease phenotype in a family member of the descendant object.

According to the method of the second aspect of the present invention, step (vii) further comprises: exclusion of a genotyping error site from within a haplotype.

In another preferred embodiment, the genotyping error site is selected from the group consisting of a genotyping error that violates the Mendelian inheritance law, violation of the positive crossover interference theory, or a combination thereof.

In another preferred embodiment, the exclusion processing of the violation of the positive crossover interference theory comprises: when two molecular marker sites in a centimorgan (cM) are experiencing double crossovers or recombined twice, the molecular markers within this recombination region are determined to be genotype typing errors.

In another preferred embodiment, each of the data sets is selected from the group consisting of a data set based on somatic cells, a data set based on an embryo culture fluid, a data set based on plasma cell-free DNA, a data set based on a sperm, a data set based on an ovum, a data set based on a polar body, or a combination thereof.

In another preferred embodiment, each of the data sets is obtained by using the same method.

In another preferred embodiment, each of the data sets is derived from detection results of the following techniques: a SNP microarray chip, a MassARRAY flight mass spectrometry chip, a MLPA multiplex ligation amplification technology, a second-generation sequencing, a third-generation sequencing, or a combination thereof.

In another preferred embodiment, each of the data sets is obtained by a method comprising the following steps:

(i) Providing nucleic acid samples derived from a descendant individual, father thereof, mother thereof and a reference object;
(ii) Performing genetic analysis (e.g. sequencing) on the nucleic acid samples to obtain genome sequencing data of the nucleic acid samples, thereby obtaining each of the data sets (i.e. a first data set, a second data set, a third data set, and data set C).

In another preferred embodiment, the nucleic acid samples are selected from the group consisting of an embryonic nucleic acid sample, a fetal nucleic acid sample, and a born offspring nucleic acid sample.

In another preferred embodiment, the nucleic acid samples are selected from the group consisting of an embryonic nucleic acid sample, a fetal nucleic acid sample, and a born human nucleic acid sample.

In another preferred embodiment, the embryonic nucleic acid sample is selected from the group consisting of a biopsy nucleic acid sample of in vitro cultured embryo, a trophoblast cell sample from blastocyst, a non-invasive nucleic acid sample such as an embryo culture fluid cell-free sample and cell-free blastocoel fluid.

In another preferred embodiment, the fetal nucleic acid sample is from amniotic fluid or umbilical cord blood.

In another preferred embodiment, the born human nucleic acid sample is derived from somatic cells, blood, plasma, sweat, urine, and semen.

In another preferred embodiment, the data set C comprises 1, 2, 3, 4 or 5 reference data sets.

According to the method of the first aspect or the second aspect of the present invention, the reference sample is selected from the group consisting of:

(Z1) an elder brother, a younger brother, an elder sister, or a younger sister of the descendant object (i.e., other descendants of the parents, including born and unborn), or a combination thereof;
(Z2) the father or mother of the father or mother of the descendant object, or a combination thereof;
(Z3) an elder brother, a younger brother, an elder sister, or a younger sister of the father or mother of the descendant object, or a combination thereof;
(Z4) an elder paternal uncle, a younger paternal uncle, a paternal aunt, a maternal uncle or a maternal aunt of the father or mother of the descendant object, or a combination thereof;
(Z5) the paternal grandfather, paternal grandmother, maternal grandfather or maternal grandmother of the father or mother of the descendant object, or a combination thereof;
(Z6) a sperm of the father of the descendant object, an ovum of the mother of the descendant object, a polar body (a first polar body or a second polar body) of the mother of the descendant object, or a combination thereof;
(Z7) any one of combinations of the Z1 to the Z6.

In another preferred embodiment, the reference objects of (Z1) and (Z6) comprise normal, abnormal mutation carriers or patients.

In another preferred embodiment, the reference objects of (Z2), (Z3), (Z4) and (Z5) are abnormal mutation carriers or patients.

In another preferred embodiment, the reference sample can be of any type, including siblings of an embryo, other family members, a single sperm, a polar body, etc. They can be analyzed by the method of the invention.

In another preferred embodiment, the first data set, the second data set, the third data set, and the data set C may contain sequencing data without abnormal mutations.

In another preferred embodiment, the molecular marker is selected from the group consisting of a SNP site, a STR polymorphic site, a RFLP site, or an AFLP site.

In another preferred embodiment, when the molecular marker is a SNP site, it can be that all sites in the preferred embodiment are heterozygous for the carrier and homozygous for the partner thereof in the SNP genotyping data.

In another preferred embodiment, the detection of the molecular markers employs a method selected from the group consisting of a SNP microarray chip, a MassARRAY flight mass spectrometry chip, a MLPA multiplex ligation amplification technology, a second-generation sequencing, a third-generation sequencing, or a combination thereof.

In another preferred embodiment, the upstream and downstream range of a target region is selected from the group consisting of: ≤1 Mbp, ≤2 Mbp, ≤3 Mbp, ≤4 Mbp, ≤5 Mbp and ≤6 Mbp region.

In another preferred embodiment, in the binary genetic vector (0,1) of step (v), the first column “0” represents the haplotype of one paternal pedigree ancestor (e.g., the paternal grandfather of the descendant object) and “1” represents the haplotype of the other paternal pedigree ancestor (e.g., the paternal grandmother of the descendant object); the second column “0” represents the haplotype of one maternal pedigree ancestor (e.g., the maternal grandfather of the descendant object) and “1” represents the haplotype of the other maternal pedigree ancestor (e.g., the maternal grandmother of the descendant object).

According to the method of the first aspect or the second aspect of the present invention, estimating a maximum possible composition of V₁, V₂, . . . Vₘ is determining a maximum probability of the ancestral haplotype composition for each individual.

In another preferred embodiment, the maximum possible composition of V₁, V₂, . . . Vₘ is estimated in step (vii) to determine whether the paternal originated haplotype is from the paternal grandfather or paternal grandmother or a combination of the paternal grandfather haplotype and the paternal grandmother haplotype, and to determine whether the maternal originated haplotype is from the maternal grandfather or maternal grandmother or a recombination combination of the maternal grandfather haplotype and the maternal grandmother haplotype.

In another preferred embodiment, if the molecular marker is a SNP site, but a genotyping error occurs indicating the site is a homozygous genotype site, then it is determined to be ADO (allele dropout).

According to the method of the second aspect of the present invention, the method further comprises step (viii): visually displaying the abnormal mutation carrying status of the haplotype of the descendant object.

In another preferred embodiment, the displaying comprises displaying the family pedigree of the individual object and the normal or abnormal haplotype composition of the corresponding individual.

In another preferred embodiment, the visualization program is written in the PERL (Practical Extraction and Report Language) scripting language.

In a third aspect the invention, a device for analyzing a haplotype of a descendant object is provided, which comprises:

(a) A data input unit which is used for inputting s data sets for the analysis, wherein s is a positive integer greater than or equal to 4, wherein the data sets are related to genome information and comprise: a first data set derived from the descendant object, a second data set derived from the father of the descendant object, a third data set derived from the mother of the descendant object, and at least one reference data set C from a reference object;
(b) An analysis site annotation unit which is used for annotating analysis sites in each of the data sets, wherein the analysis sites are molecular markers identified by analysis and detection upstream and downstream regions of a predetermined target site;
(c) A haplotype analysis unit configured to perform the following operations:
(Y1) Determining a binary genetic vector of (0, 1) for each analysis site in each of the data sets;
(Y2) Determining a maximum likelihood estimation value L by Formula Q1 using a Hidden Markov model:

$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$

wherein:
m represents the number of molecular markers upstream and downstream of each target site;
P(V₁) represents a priori value of a genetic vector;
P(Vᵢ|Vᵢ₋₁) represents a transition probability of the haplotype status between two adjacent sites;
Gᵢ represents an observed value of a genotype at the ith site;
P(Gᵢ|Vᵢ) represents an emission probability of a haplotype status;
(Y3) Determining the haplotype (or the haplotype genetic flow) of the descendant object by estimating the maximum possible composition of V₁, V₂, . . . Vₘ through a Viterbi dynamic programming algorithm; and
   (d) An output unit for outputting the analysis result of the haplotype analysis unit.

In another preferred embodiment, the analysis device further comprises one or more units selected from the group consisting of:
(e) A sequencing unit for sequencing a nucleic acid sample to obtain genomic sequence data;
(f) A quality control unit for quality control of the molecular marker genotype data; and
(g) A genotype error processing unit for exclusion of genotype errors for a haplotype in each of the data sets.

In another preferred embodiment, the analysis site is selected from the group consisting of: an abnormal mutation, a mutation site, a disease site, a kinship related site or a combination thereof.

It should be understood that, within the scope of the invention, each technical feature above in the invention and each technical feature particularly described below (such as in Examples) can be combined with each other to constitute a new or preferred technical solution. Due to space limitations, they will not be reiterated herein.

### BRIEF DESCRIPTION OF THE FIGURES

FIG. 1 shows a schematic diagram of the family genetic pedigree and haplotype genetic flow. The schematic diagram is a pedigree map for an autosomal recessive genetic disease. A square represents male; a circle represents female; a diamond represents unknown sex (an embryo); an empty indicates that a person is normal; a half-empty indicates a heterozygous carrier of a pathogenic mutation; a solid indicates diseased; left slashes on the bar indicate carrying of maternal origin, right slashes on the bar indicate carrying of paternal origin; the leftmost blue molecular markers indicate the upstream of the pathogenic mutation region and the leftmost oranges indicate the downstream of the pathogenic mutation region; the numbers of the second column on the left indicate the genetic distances, which can excellently show the probability of chromosomal crossover between different molecular markers.

FIG. 2 shows a schematic diagram of haplotype analysis of a case having autosomal dominant inheritance in one embodiment of the invention.

FIG. 3 shows the inference results of the pathogenic site carrying status in three embryos in Example 1 of the invention.

FIG. 4 shows the inference results of the carrying status of balanced translocation in 5 embryos having normal chromosomal copy number in Example 2 of the invention.

FIG. 5 shows analysis results of a haplotype in Example 3 of the invention, which can be used to infer the carrying status of a single-gene disease-related site.

FIG. 6 shows analysis results of a haplotype based on sequencing data of amniotic fluid in Example 4 of the invention, wherein the target site is a single-gene disease-related site.

FIG. 7 shows the analysis results of the haplotype of an object to be analyzed in one embodiment. A born child of the male partner and the female partner, i.e., a priori, is used as the reference object.

FIG. 8 shows the analysis results of the haplotype of an object to be analyzed in one embodiment, wherein the father of the male partner and the father of the female partner are reference objects, while other objects of an embryo's family, such as the mother of the male partner and the mother of the female partner, are used in an optimized embodiment instead of as essential objects.

FIG. 9 shows the analysis results of the haplotype of an object to be analyzed in one embodiment, wherein the elder sister of the female partner and the father of the female partner are reference objects, while other objects of an embryo's family, such as the younger sister of the female partner is used in an optimized embodiment instead of as an essential object.

FIG. 10 shows the analysis results of the haplotype in one embodiment of the invention.

FIG. 11 shows the analysis results of the haplotype of an object to be analyzed in one embodiment (FYGCE1_1 and FYGCE2_1). If there is no information on the father and mother of the male partner, the maternal grandmother of the male partner can also be used as the reference object.

FIG. 12 shows the analysis results of the haplotype of an object to be analyzed in one embodiment, wherein, any single sperm, i.e. danjingzil1 and danjingzil8 may be used as the reference object.

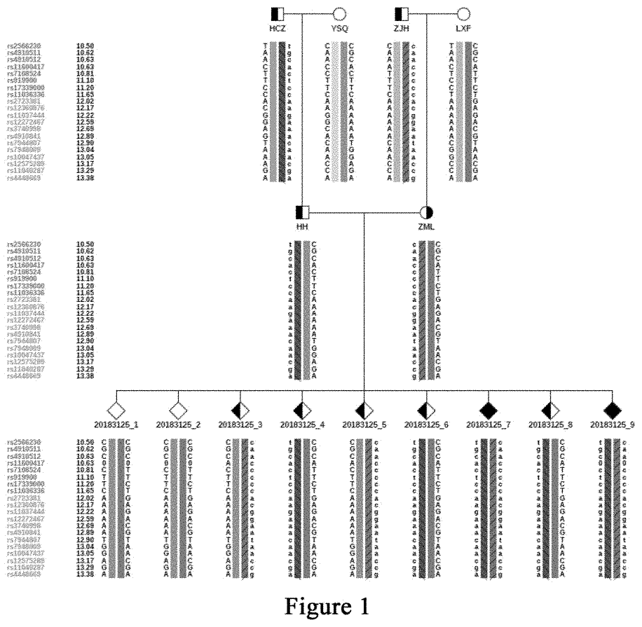

In FIGS. 1-12, the unit of each value is the genetic distance centimorgan (cM) or the relative chromosomal physical distance from the target site.

### DETAILED DESCRIPTION OF EMBODIMENTS OF THE INVENTION

To address the drawbacks in the prior art, the invention has unexpectedly developed, for the first time, an analysis method and a device that can be used for an accurate and efficient determination of the haplotype of a descendant object after extensive and intensive research. The method of the invention uses data sets of a number of or all family members of the descendant object for analysis, thus allowing more efficient and accurate haplotype analysis results, particularly suitable for haplotype analysis in a case of incomplete pedigree information. The invention is accomplished on this basis.

The invention can be used to analyze the haplotype, genetic flow, and/or kinship of a descendant object, for example, to analyze the haplotype of the descendant object in an abnormal mutation carrying status.

### Terms

As used herein, the terms “method of the invention”, “analysis method of the invention for determining a haplotype of a descendant object”, and “data analysis method of the invention for determining a haplotype genetic flow” can be used interchangeably, referring to the method described in the first aspect and/or the second aspect of the invention.

### Analysis Method for Determining a Haplotype of a Descendant Object

The invention provides an analysis method for determining a haplotype of a descendant object (such as an embryo, a fetus or a born descendant) in an abnormal mutation carrying status.

Particularly, in the invention, the haplotype compositions of all pedigree members are analyzed using the Lander-Green algorithm according to the theory of gene linkage and crossover as well as the genetic information of all pedigree members (e.g., it can be determined whether the two haplotypes of each individual are most likely to be inherited from the paternal grandfather or grandmother, and from the maternal grandmother or grandfather), thus the vertical transfer of gene flow in the whole pedigree is clearly shown (see FIG. 1); next, the abnormal haplotype is inferred by combining the abnormal mutation carrying status of a known object (e.g., father, mother or a reference object) with the haplotype composition thereof; finally, the abnormal mutation carrying status of a descendant object (e.g., an embryo, a fetus or a born descendant) is inferred according to whether an abnormal haplotype is inherited by the descendant object (e.g., an embryo, a fetus or a born descendant).

Typically, a particular technical solution of the method is as follows:

1) Desired objects: a single-cell amplification product of a descendant (e.g., an embryo, a fetus or a born descendant), parental nucleic acid objects, and a nucleic acid object of a sibling or other family member of a descendant (e.g., an embryo, a fetus or a born descendant), either diseased or normal.
2) Detecting molecular markers within a certain range upstream and downstream of a target abnormal mutation region. The molecular markers are not limited to polymorphic sites such as STR and SNP; the detection means can be whole-genome sequencing, targeted sequencing (amplicon sequencing), or gene chip; the upstream and downstream range of the target region can be 1 Mbp, 2 Mbp, 3 Mbp or even a whole chromosome.
3) Performing corresponding quality control for the molecular marker genotype data, for example, quality control for single-cell whole-genome amplification efficiency, identification of Mendelian inheritance violation, etc.
4) Polymorphic site selection criteria: in SNP genotyping data, said site is heterozygous for the carrier and homozygous for the partner thereof; if it is microsatellite STR and other polymorphic types (polymorphic types are more abundant), there is no such a restriction.
5) For each polymorphic site (i.e., a polymorphic site (e.g., a SNP site)) in each object (or a sample of an object, or a data set of an object), constructing a binary genetic vector of (0, 1). The first column “0” indicates the haplotype of one paternal ancestor such as the paternal grandfather of the object, and “1” indicates the haplotype of the other paternal ancestor such as the paternal grandmother of the object; the second column “0” indicates one maternal ancestor such as the maternal grandfather of the object, and “1” indicates the haplotype of the other maternal ancestor such as the maternal grandmother of the object. n objects constitute 2n vectors of Vᵢ, wherein i represents a site. Wherein Vi is a Hidden Markov Chain state.
6) Constructing a maximum likelihood estimation by the following Formula using a Hidden Markov Model strategy.

$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$

Wherein, m represents the number of sites; P(V₁) represents a priori value of a genetic vector; P(Vᵢ|Vᵢ₋₁) represents a transition probability of the haplotype status between two adjacent sites, calculated by using a genetic map and obtaining a recombination rate; Gᵢ represents an observed value of a genotype at the ith site; P(Gᵢ|Vᵢ) represents an emission probability of a haplotype status, which is calculated by combining an observed value of the genotype of the object and a genotype of an ancestor thereof, and using the Mendelian inheritance law.

7) Estimating a maximum possible composition of V₁, V₂, . . . Vₘ, i.e., a maximum probability of the ancestral haplotype composition for each individual, by using a Viterbi dynamic programming algorithm, to determine whether the paternal originated haplotype is from the paternal grandfather or the paternal grandmother, and whether the maternal originated haplotype is from the maternal grandfather or the maternal grandmother.
8) Handling error genotypes within haplotypes: In addition to the occurrence of obvious genotype errors that violate the Mendelian inheritance law, another principle for identifying error genotypes is that if two crossovers or two recombinations exist between two molecular markers within a centimorgan (cM), then it appears that a genotype error takes place at the molecular markers within this recombination region. Taking a SNP site as an example, if this site is genotyped as homozygous, then the result is interpreted as ADO, while if this site genotyped as heterozygous, then the result is interpreted as having another genotype error.
9) Next, inferring an abnormal haplotype based on the disease phenotype information of known family members (father, mother and reference objects).
10) Determining whether or not the descendant (e.g., an embryo, a fetus, or a born descendant) carries an abnormal haplotype according to the haplotype genetic flow direction of the descendant (e.g., an embryo, a fetus, or a born descendant), thereby the abnormal mutation carrying status of the descendant (e.g., an embryo, a fetus, or a born descendant) is inferred.
11) Finally, clearly displaying the family pedigree and the normal or abnormal haplotype composition of each individual by running a visualization program written in the PERL (Practical Extraction and Report Language) scripting language.

Typically, objects of interest analyzed in the invention are: a descendant object+the father and/or the mother of the descendant object+at least one other relative of the descendant object (referred to as a reference object hereinafter). Additionally, the sample of the descendant object can be from an embryo, a fetus, blood, culture fluids or a born human (somatic cells).

In the invention, the analysis site comprises (but is not limited to): an abnormal mutation, a mutation site, a disease site, a kinship related site, or a combination thereof.

### Reference Object

In the invention, the reference object is one or more other relatives (other than the parents) of the object to be tested (i.e., the descendant object). A minimum of at least one reference object is required, and the more reference objects there are, the higher the accuracy of the inference is, which belongs to a preferred technical embodiment.

Typically, the reference object can be selected from one or more of the following 6 scenarios, wherein the father and mother of the object to be tested are hereinafter referred to as “the male partner and the female partner”:

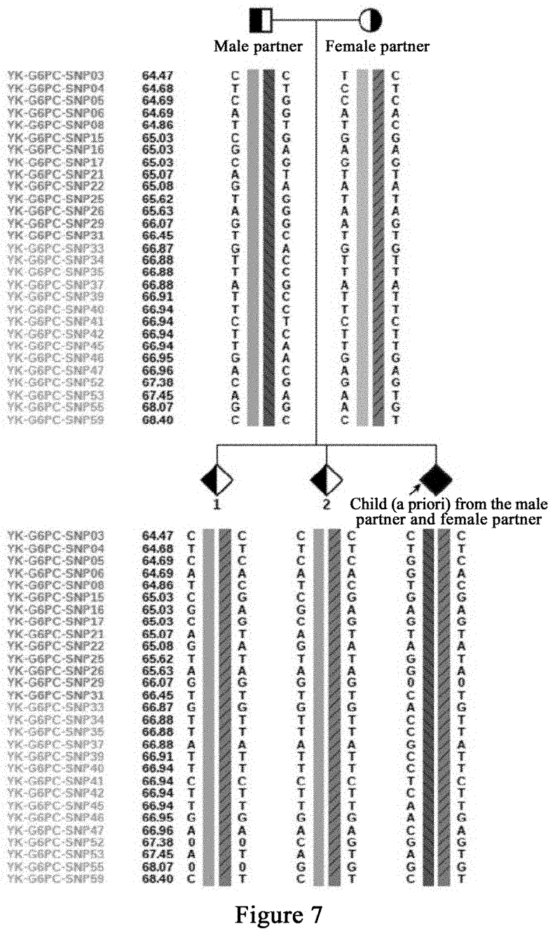

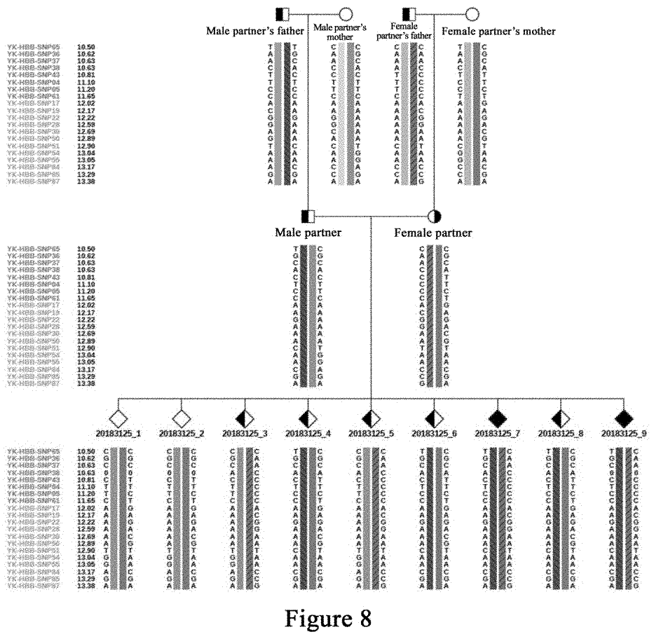

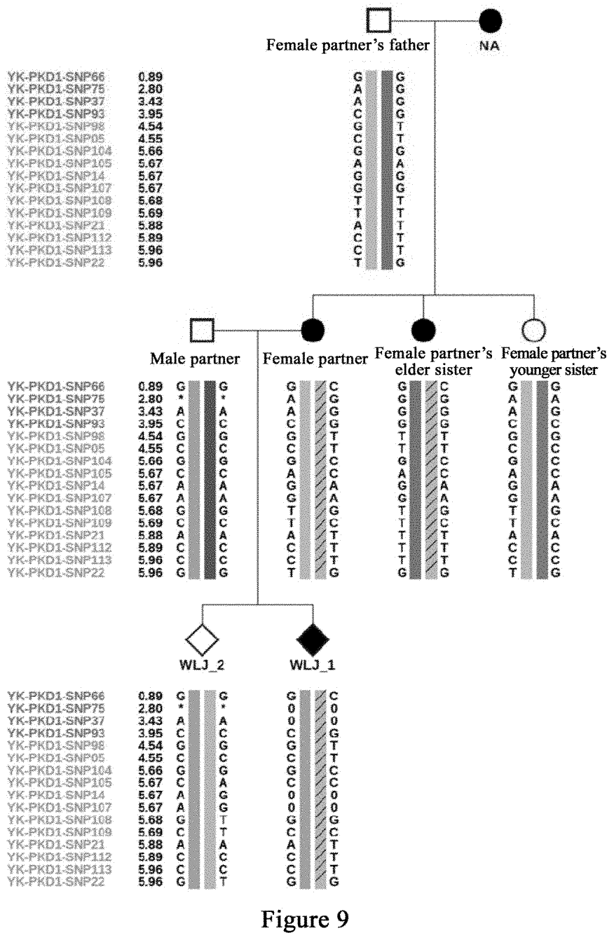

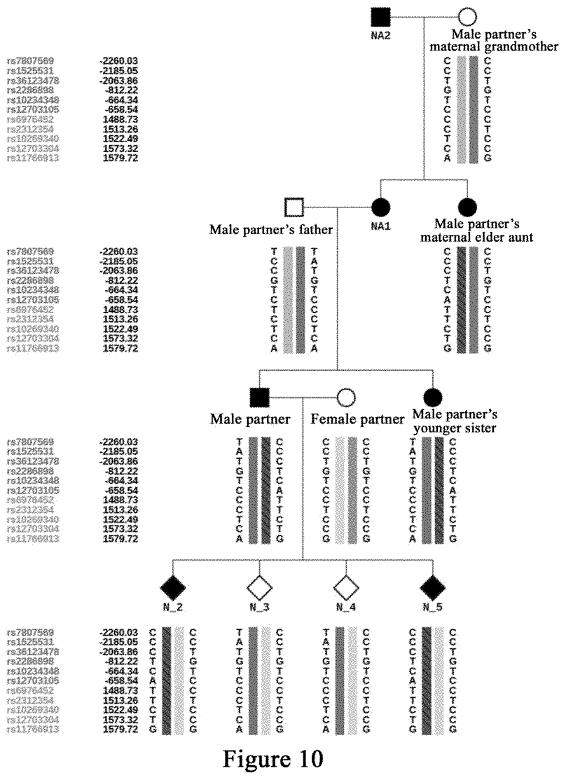

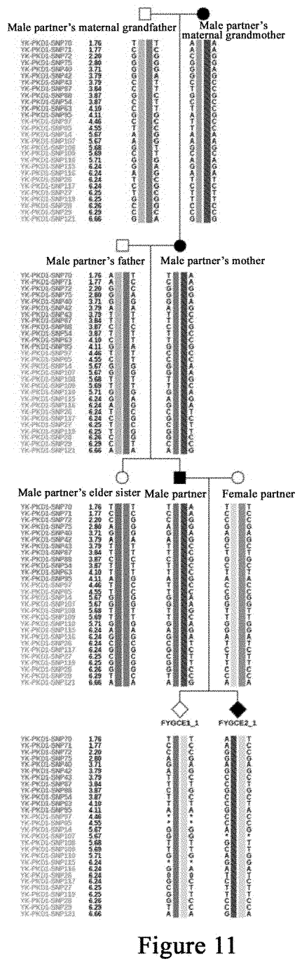

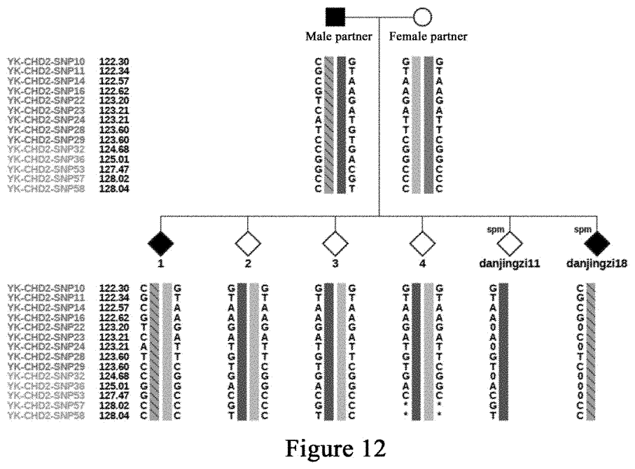

1) Only an offspring of the male partner and the female partner is used as a reference object (which is healthy or diseased). Particularly, the offspring can be a born child of the male partner and the female partner (i.e., an elder brother, a younger brother, an elder sister or a younger sister of the object to be tested); it can also be an unborn child of the male partner and the female partner, such as amniotic fluid, umbilical cord blood, aborted fetus, etc.; it can also be an embryo having an identified disease phenotype. See FIG. 7 for a representative example.
2) Only the parents, either of whom is a carrier or a patient of the pathogenic site, of the male partner and the female partner are used as reference objects. If the male partner is the carrier or the patient of the pathogenic site, then the reference object can be a parent of the male partner, who shall also be a carrier or a patient of the pathogenic site (to ensure that the pathogenic site is inherited instead of a new mutation); similarly, if the female partner is the carrier or the patient of the pathogenic site, then the reference object can be a parent of the female partner, who shall also be a carrier or a patient of the pathogenic site (to ensure that the pathogenic site is inherited instead of a new mutation). See FIG. 8 for a representative example.
3) Only the elder brother, younger brother, elder sister or younger sister, who is also a carrier or a patient of the pathogenic site, of the male partner and the female partner is used as a reference object. One of the elder brother, younger brother, elder sister or younger sister of the pathogenic site carrier or patient is sufficient, provided that he/she is also a carrier or a patient of the pathogenic site (to ensure inheritance, excluding the possibility of a new mutation). Meanwhile, it is also preferable to have the information from a parent of the pathogenic site carrier or patient, with no restrictions on the phenotype state of the disease. See FIG. 9 for a representative example.
4) Only the younger paternal uncle, older paternal uncle, paternal aunt, maternal uncle or maternal aunt of the male partner and the female partner, who is also a carrier or a patient of the pathogenic site, is used as a reference object. One of the younger paternal uncle, older paternal uncle, paternal aunt, maternal uncle or maternal aunt of the pathogenic site carrier or patient is sufficient, provided that he/she is also a carrier or a patient of the pathogenic site (to ensure inheritance, excluding the possibility of a new mutation). See FIG. 10 for a representative example.
5) Only the paternal grandfather and paternal grandmother, or maternal grandfather and maternal grandmother (also a carrier or a patient of the pathogenic site) of the male partner and the female partner are used as the reference objects. The paternal grandfather and paternal grandmother, or maternal grandfather and maternal grandmother of the pathogenic site carrier or patient are sufficient, provided that he/she is also the carrier or the patient of the pathogenic site (to ensure inheritance, excluding the possibility of a new mutation). See FIG. 11 for a representative example.
6) If none of the above objects is suitable as a reference object, a single sperm from the male partner or a polar body from the female partner (either normal or a carrying the pathogenic site) can be used as a reference object. If the male partner is the carrier or the patient of the pathogenic site, a single sperm thereof may be used as a reference object, regardless of the carrying status of the pathogenic mutation; if the female partner is the carrier or the patient of the pathogenic site, the first polar body or the second polar body thereof may be taken as a reference object. See FIG. 12 for a representative example.

### Device for Analyzing a Haplotype of a Descendant Object

The present invention also provides an analysis device (or analysis system) for performing the method of the invention. Typically, the analysis device comprises:

(a) A data input unit which is used for inputting s data sets for the analysis;
(b) An analysis site annotation unit which is used for annotating analysis sites in each of the data sets;
(c) A haplotype analysis unit configured to perform the following operations:
(Y1) Determining a binary genetic vector of (0, 1) for each analysis site in each of the data sets;
(Y2) Determining a maximum likelihood estimation value L by Formula Q1 using a Hidden Markov model:

$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$

(Y3) Determining the haplotype (or the haplotype genetic flow) of the descendant object by estimating the maximum possible composition of V₁, V₂, . . . Vₘ through a Viterbi dynamic programming algorithm; and
   (d) An output unit for outputting the analysis result of the haplotype analysis unit.

Additionally, the analysis device further comprises:

(e) A sequencing unit for sequencing a nucleic acid sample to obtain genomic sequence data;
(f) A quality control unit for quality control of the molecular marker genotyping data; and
(g) A genotype error processing unit for exclusion of genotype errors for a haplotype in each of the data sets.

In the present invention, the output unit can be a printer, a display or other output devices.

### Main Advantages of the Invention

(1) The method of the present invention uses the genetic information of a number of or all samples in the family to carry out haplotype analysis. The haplotype phasing is more accurate as the haplotype analysis is based on an optimized formula and algorithm;
(2) The method of the present invention can even use the genetic information from a number of objects to back infer the haplotype of the parental carrier, so that the a triple heterozygous site (for example, a heterozygous site carried by a parent and his/her parents) can be successfully processed and haplotyping, thus there are more informative sites to deal with cases where the reference object is not a sibling of the embryo, resulting in more reliable results.
(3) The method is convenient and flexible, and any type of reference objects, including siblings of an embryo, other family members, a single sperm, a polar body, etc. can be analyzed by the method. For single-gene genetic diseases, the method can flexibly handle diseases having different inheritance patterns, such as autosomal dominant inheritance, autosomal recessive inheritance, X-chromosome-linked inheritance, etc.
(4) The method of the present invention is particularly suitable for cases where the pedigree information is incomplete, and is substantially capable of successfully obtaining haplotype analysis results.

### EXAMPLES

The following specific Examples further illustrate the present invention. It should be understood that these Examples are only used to illustrate the present invention and not to limit the scope of the present invention. The experimental method without describing specific conditions in the following Examples, is usually performed according to conventional conditions, such as those in Sambrook and Russell et al. (Molecular Cloning—A Laboratory Manual, (Third Edition) (2001) CSHL Press), or as recommended by the manufacturer. Percentages and parts are by weight unless otherwise indicated. The experimental materials and reagents used in the following Examples are all commercially available unless otherwise specified.

### Example 1: Embryo Biopsy Sample+Inference of the Carrying Status of the Embryo Having Single-Gene Disease-Related Site

1) Pedigree information: methylmalonic acidemia, an autosomal recessive genetic disease with the pathogenic gene MUT, the male partner being a carrier of MUTc.323G>A mutation, the female partner being a carrier of MUTc.729_730insTT mutation, and the aborted fetus derived from the male partner and the female partner being a carrier of the paternal mutation. Three embryo samples were to be tested.
2) Trophoblast cells from the embryonic blastocyst were extracted, gDNAs of the father and the mother of the embryos (referred to as the male partner and the female partner hereinafter), as well as the gDNA of the other aborted fetus of the parents of the embryos, were extracted.

Several trophoblast cells from the blastocyst were directly placed in a 5 ul lysis solution, for single-cell whole-genome amplification by MALBAC two-step method (Universal Sample Processing Kit for Gene Sequencing, Xukang Medical Technology (Suzhou) Co., Ltd., Cat. No. XK-028).

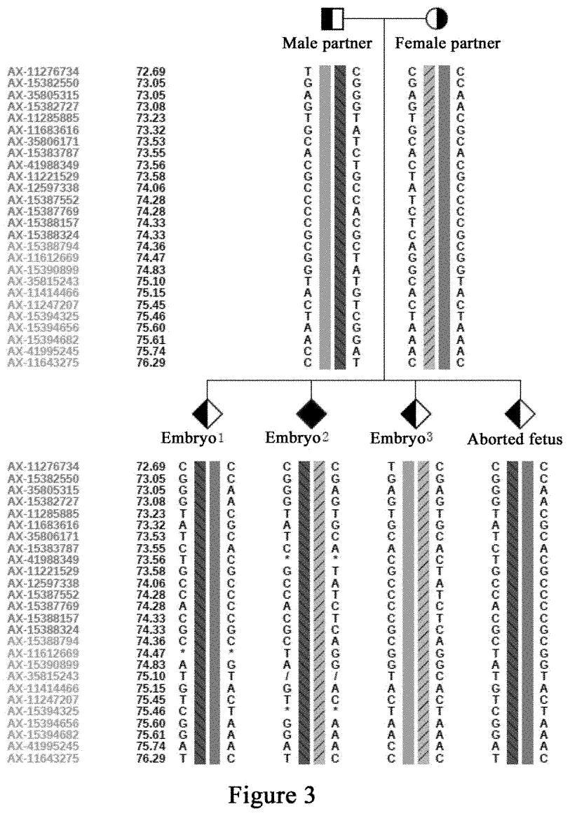

3) For the gDNAs of the male partner, the female partner and the aborted fetus, and the embryo whole-genome amplification products, genotyping detections were carried out using the PMRA (Axiom Precision Medicine Research Array) chip of Thermo Fisher Scientific.
4) After the chip scan data were obtained, genotyping analysis was performed using the Genotyping functional module in the Axiom Analysis Suite analysis platform of Thermo Fisher Scientific.
5) The quality control standards for genotype data were as follows:
\textcircled{1} Sites with >65% call rates at the sample level and genotype quality meeting criteria of PolyHighResolution, NoMinorHom, MonoHighResolution and Hemizygous were used for subsequent analysis. All 3 embryos to be tested and the family members met the quality control standard.
\textcircled{2} Considering the uneven feature in single-cell whole-genome amplification efficiency, the quality control of MALBAC amplification efficiency was performed on embryonic amplification products. Based on the constructed reference sample system (BAM sequencing file library) for MALBAC amplification products by our company, sites with absolute sequencing depth greater than the average genomic sequencing depth were selected out for analysis in the next step.
\textcircled{3} Genotypes with Mendelian violations were identified according to the Law of Mendelian inheritance segregation. Since the Mendelian inheritance law was not violated by all embryos at a site, the site would be retained, but the site where Mendelian violation occurred on the genotypes of embryos would be marked as defective data.
   6) SNP site information within 2 Mbp upstream and downstream of the MUT gene was extracted, and a total of 15 upstream sites and 11 downstream sites related to those heterozygous for the male partner and homozygous for the female partner, or those heterozygous for the female partner and homozygous for the male partner were selected for further analysis.
   7) Each of the sites was ordered by its position on the chromosome, and a binary genetic vector of the site was constructed as Vᵢ=(p_{1,i}, m_{1,i}, p_{2,i}, m_{2,i}, p_{3,i}, m_{3,i}) wherein i was a value from 1 to 16. For example, at the No. 1 site, AX-11643275 (see FIG. 3), the genotype was C/T for the male partner and C/C for the female partner, and the priori haplotype genetic vector V₁ of the 3 embryos was (1,1,1,0,0,0,1,1). The transition matrix P(Vᵢ|Vᵢ₋₁) was the transition probability of the haplotype status between two sites, estimated by the chromosomal recombination rate, wherein recombination meant changes in haplotype status. The recombination rate was calculated by the genetic distance between the two sites on the genetic map, for example, a genetic distance of 1cM represented 1% recombination rate. The genetic map used the data from 1000 genomes phase 3. The emission matrix P(Gᵢ|Vᵢ) output the probability of the currently observed genotype after the haplotype genetic vector at the site was given according to the Mendelian inheritance law. The formula for calculation of the maximum probability of the 16-site haplotype compositions was as follows.

$$L = {\sum }_{{V}_{1}} … {\sum }_{{V}_{16}} P ( {V}_{1} ) {\prod }_{i = 2}^{16} P ( {V}_{i} \vert {V}_{i - 1} ) {\prod }_{i = 1}^{16} P ( {G}_{i} \vert {V}_{i} ) \qquad ( Q2 )$$

8) The ancestral haplotype composition of the maximum probability was estimated for the 3 embryos having V₁, V₂, . . . . V₁₆ (two haplotypes for each embryo) using a Viterbi dynamic programming algorithm.
9) After the haplotype was constructed, the pathogenic mutation carrying haplotype could be distinguished from the normal haplotype based on the phenotypic information of the aborted fetus and of the male partner and the female partner (FIG. 3). If a pathogenic mutation of paternal origin was inherited by the aborted fetus, then a dark blue haplotype was inherited from the father, and this dark blue haplotype indicated carrying the pathogenic mutation; if the pathogenic mutation of maternal origin was not carried by the aborted fetus, then the dark orange haplotype inherited from the mother was a normal haplotype, while the light orange haplotype indicated carrying the pathogenic mutation (because the mother was also a heterozygous carrier of the pathogenic mutation). The carrying status of the pathogenic mutation in the embryos could be determined by its inherited haplotype. Therefore, it was inferred that, embryo 1 was a carrier of paternal mutation (inheritance of the dark blue haplotype), embryo 2 was a carrier of parental and maternal mutations (inheritance of the dark blue and light orange haplotype) which would lead to the disease phenotype, and embryo 3 was a carrier of maternal mutation (inheritance of the light orange haplotype). For easy distinction, the haplotype carrying maternal pathogenic mutation was marked with left slashes, while the haplotype carrying paternal pathogenic mutation was marked with right slashes.
10) Embryo amplification products were simultaneously screened for embryo chromosomal aneuploidy using CNV-Seq, and it was found that embryo No. 1 had no abnormal chromosomal copy number, while the other two embryos had abnormal chromosomal copy number (Table 1).
11) First-generation Sanger sequencing verification: embryo No. 1 carried paternal mutation, embryo No. 2 carried parental and maternal compound heterozygous mutation, and embryo No. 3 carried maternal mutation, which were consistent with the results of SNP haplotype analysis (Table 1).
12) Since the disease was autosomal recessive, heterozygous carrier would not lead to a clinical disease phenotype. In the absence of a completely normal embryo for transfer, the paternal-mutation carrier, embryo No. 1, was transferred after obtaining the consent of the male partner and the female partner, and the female partner successfully conceived. The results of amniotic fluid testing in the mid-trimester and umbilical cord blood testing during delivery confirmed the correctness of the PGT detection results.

TABLE 1
Summary of the detection results in Example 1

| Sample | Aneuploidy detection | Analysis result of pathogenic site |
|---|---|---|
| name | result | and SNP linkage |
| Embryo 1 | 46, XN | Paternally carried |
| Embryo 2 | 48, XN, +21(×4) | Paternally carried and maternally |
| | | carried |
| Embryo 3 | 45, XN, −21(×1) | Maternally carried |

### Example 2: Embryo Biopsy Sample+Inference of Chromosomal Balanced Translocation Carrying Status in Embryos

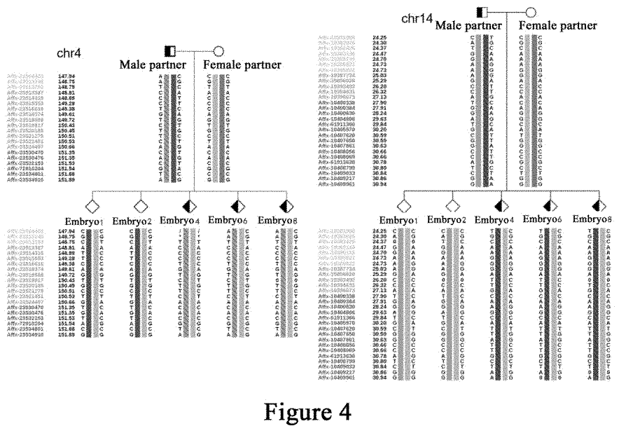

1) Pedigree carrying chromosomal balanced translocation (reciprocal translocation): carried by male partner, with karyotype of 46, XY, t(4;14)(q31.1;q21), female partner was normal, 9 embryos were to be tested.
2) The peripheral blood gDNAs of the male partner and female partner were extracted. The embryo samples were trophoblast cells from blastocysts. Thermal lysis, then single-cell whole-genome amplification by MALBAC two-step method as described in Example 1 were performed.
3) Embryo amplification products were subjected to CNV-Seq detection for chromosomal aneuploidy. The detection results showed that embryos 1, 2, 4, 6, and 8 were CNV normal, while embryos 3, 5, and 7 were CNV abnormal (Table 2).
4) Embryos No. 3 and No. 7 having abnormal CNVs were used to determine the breakpoint, see patent CN106834490A for the specific method.
5) Genotyping detections were carried out for the gDNAs of the male partner, the female partner, and the whole-genome amplification products of the embryo Nos. 1, 2, 3, 4, 6, 7 and 8 using the PMRA chip of Thermo Fisher Scientific.
6) The quality control standards were as described in Example 1.
7) The haplotype analysis was performed for embryo Nos. 3 and 7 with unbalanced CNVs as reference samples, and for the male partner, the female partner and other embryos with normal CNVs (embryo No. 1, embryo No. 2, embryo No. 4, embryo No. 6 and embryo No. 8). For the specific steps of the haplotype analysis, refer to steps 7 and 8 of Example 1.
8) Based on the segregation law of the quadriradial structures of chromosomes with balanced translocations, which were formed during meiosis, the chromosomes with the haplotypes within 3M upstream of the breakpoints of chromosome 4 of embryo No. 3 and of chromosome 14 of embryo No. 7 were translocation chromosomes; and the chromosomes with the haplotypes within 3M upstream of the breakpoints of chromosome 14 of embryo No. 3 and of chromosome 4 of embryo No. 7 were normal chromosomes. The haplotypes of other CNV normal embryos in this region could be compared with these two embryos to determine whether the embryo was a normal embryo or an embryo carrying chromosomal balanced translocation (FIG. 4).

See Table 2 for the inference results.

TABLE 2
Summary of detection results in Example 2

| Sample | | Translocation carrying |
|---|---|---|
| name | Chromosomal aneuploidy detection | detection result |
| Embryo 1 | 46, XN | No carrying |
| Embryo 2 | 46, XN | No carrying |
| Embryo 3 | 47, XN, −4q(q31.21→q33, ~29M, ×1), −4q | — |
| | (q34.1→q35.2, ~19M, ×1), +14q | |
| | (q13.1→q32.33, ~72M, ×3), +21(×3) | |
| Embryo 4 | 46, XN | Carrying |
| Embryo 5 | 45, XN, −22(×1) | — |
| Embryo 6 | 46, XN | Carrying |
| Embryo 7 | 46, XN, −2q(q24.3→q37.3, ~76M, ×1, | — |
| | mos, ~40%), +4q(q31.21→q35.2, ~49M, ×3), −14q | |
| | (q13.1→q32.33, ~72M, ×1) | |
| Embryo 8 | 46, XN | Carrying |

### Example 3: Blastocyst Culture Fluid Sample+Inference of Carrying Status of the Embryo Having Single-Gene Disease-Related Site

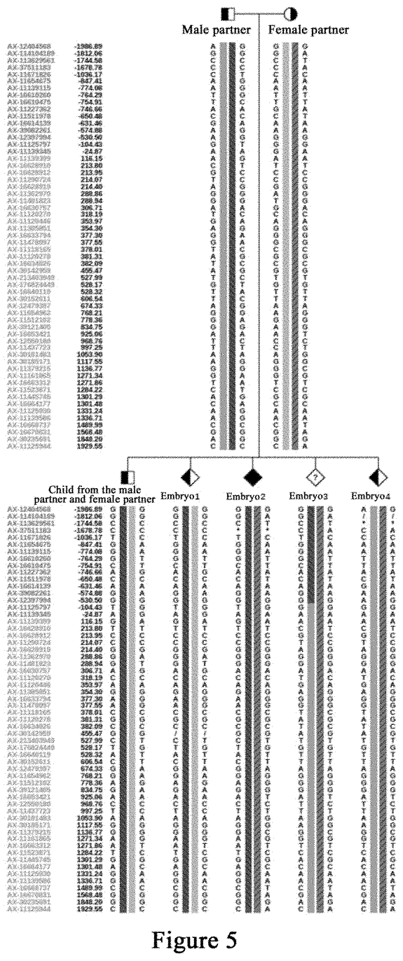

(1) Pedigree information: β-thalassemia, an autosomal recessive genetic disease with the pathogenic gene HBB, the male partner being a carrier of HBB IVS-II-654C>T mutation, the female partner being a carrier of the same HBB IVS-II-654C>T mutation, and the child of the male partner and the female partner being a carrier of a heterozygous mutation. 4 embryo samples were to be tested. In this case, because it was impossible to determine whether the heterozygous mutation carried by the child of the male partner and the female partner was from the male partner or the female partner, the pathogenic haplotype of the male partner or the female partner could not be determined at the stage, but had to be inferred from the verification results of the first-generation sequencing of the embryos.
(2) The cell-free blastocyst culture fluids of 4 in-vitro embryos cultured to the 5th day were taken as the test samples. The gDNAs of the father and mother of the embryo, referred to as the male partner and the female partner hereinafter, as well as the gDNA of another born child of the male partner and the female partner, were extracted. 5 ul of the blastocyst culture fluid was subjected to thermal lysis, then a single-cell whole-genome amplification by MALBAC two-step method (Universal Sample Processing Kit for Gene Sequencing, Xukang Medical Technology (Suzhou) Co., Ltd., Cat. No. XK-028).
(3) First-generation Sanger sequencing verification: embryo sample No. 2 clearly carried paternal and maternal mutations; embryo sample No. 1 carried a heterozygous mutation which could not be determined to be of paternal or maternal origin; embryo sample No. 3 carried a heterozygous mutation, which could not be determined to be of paternal or maternal origin; and embryo sample No. 4 carried a heterozygous mutations, which could not be determined to be of paternal or maternal origin.
(4) Since embryo sample No. 2 clearly carried paternal and maternal mutations, embryo sample No. 2 was used as a reference sample for the inference of abnormal haplotypes as of paternal or maternal origin.
(5) Genotyping detections were carried out for the gDNAs of the male partner, the female partner and the child thereof, and the embryo whole-genome amplification products using the PMRA (Axiom Precision Medicine Research Array) chip of Thermo Fisher Scientific.
(6) After the chip scan data were obtained, genotyping analysis was performed using the Genotyping functional module in the Axiom Analysis Suite analysis platform of Thermo Fisher Scientific.
(7) The quality control standards for genotype data were as described in Example 1.
(8) SNP site information within 2 Mbp upstream and downstream of the HBB gene was extracted, and a total of 15 upstream sites and 11 downstream sites related to those heterozygous for the male partner and homozygous for the female partner, or those heterozygous for the female partner and homozygous for the male partner were selected out for further analysis.
(9) Refer to Example 1 for the haplotype analysis method. The analysis results were shown in FIG. 5.
(10) After the haplotype was constructed, the pathogenic mutation carrying haplotype could be distinguished from the normal haplotype based on the phenotypic information of the child from the male partner and the female partner and the descendant object 2 (FIG. 5 and Table 3).
(11) Embryo amplification products were simultaneously screened for embryo chromosomal aneuploidy by CNV-Seq. The results were shown in Table 3. All embryos had CNV abnormalities.
(12) Therefore, no normal embryo was available for transfer.

TABLE 3
Summary of detection results in Example 3

| Sample | Aneuploidy detection | Analysis result of pathogenic site |
|---|---|---|
| name | result | and SNP linkage |
| Sample 1 | 46, XX, −Xq(q21.31→q21.32, ~4.7M, ×1), +6p | Paternally carried |
| | (p22.1→p21.2, ~7.9M, ×3), −6q(q27→qter, ~5.4M, ×1), −9q | |
| | (q13→q21.11, ~4.7M, ×1), −10q(q26.2→qter, ~5.6M, ×1), +12q | |
| | (q13.11→q14.1, ~9.6M, ×3), +16p(×3, mos, ~30%), +17q | |
| | (q21.2→q21.33, ~9.4M, ×3), −18q(q22.3→qter, ~5.3M, ×1), +19p | |
| | (×3, mos, ~50%), +19q(q13.11→q13.43, ~24M, ×3), −21p | |
| | (×1), +22q(q12.3→q13.2, ~7.6M, ×3) | |
| Sample 2 | 46, XN, +1q(q21.3→q23.3, ~8.1M, ×3), −2p | Paternally carried |
| | (p25.3→p25.1, ~6.6M, ×1), −2q(q35→q37.3, ~18M, ×1), −5p | and maternally |
| | (pter→p15.31, ~9.1M, ×1), −7q(q36.2→qter, ~5.9M, ×1), −9q | carried |
| | (q13→q21.11, ~4.7M, ×1), −10q(q26.2→qter, ~6.6M, ×1), +12q | |
| | (q13.13→q14.1, ~6.6M, ×3), +17p(×3, mos, ~40%), −18p | |
| | (×1, mos, ~40%), −18q(q22.3→qter, ~5.3M, ×1), +19p | |
| | (p13.3→p13.11, ~18.5M, ×4), +19q | |
| | (q13.12→q13.43, ~23M, ×3), −21p(×1) | |
| Sample 3 | 46, XN, +1q(q21.3→q23.3, ~8.3M, ×3), +6p | Maternally |
| | (p22.1→p21.2, ~7.8M, ×3), −9q(q13→q21.11, ~4.7M, ×1), −10q | carried (Paternal |
| | (q26.2→qter, ~6.0M, ×1), −13q(q31.1→q34, ~31M, ×1, | chromosomal |
| | mos, ~30%), +17p(p13.3→p13.1, ~9.8M, ×3), −18q | crossover) |
| | (q22.3→qter, ~5.4M, ×1), +19p(p13.3→p13.2, ~7.9M, ×4), +19p | |
| | (p13.2→p13.11, ~7.1M, ×3), + 19q(q13.13→q13.2, −4.7M, ×4), +19q | |
| | (q13.32→q13.41, ~4.4M, ×4), −21p(×1) | |
| Sample 4 | 46, XN, +6p(p22.1→p21.2, ~7.8M, ×3), −6q | Maternally |
| | (q27→qter, ~4.3M, ×1), −9q(q13→q21.11, ~4.7M, ×1), +11q | carried |
| | (q12.2→q13.2, ~5.7M, ×3), −15q(q11.1→q13.1, ~10.0M, ×1), +17p | |
| | (p13.3→p13.1, ~9.4M, ×3), +17q(q21.2→q21.33, ~9.5M, ×3), +17q | |
| | (q24.3→qter, ~10.8M, ×3), +19p(p13.3→p13.2, ~7.7M, ×4), +19p | |
| | (p13.2→p13.11, ~7.2M, ×4), +19q(q13.11→q13.43, ~24M, ×3), +20q | |
| | (q11.21→q11.23, ~7.9M, ×3), +20q(q13.11→q13.2, ~8.9M, ×3), −21p | |
| | (×1), +22q(q12.3→q13.31, ~10.0M, ×3) | |

### Example 4: Amniotic Fluid+Inference of the Carrying Status of a Single-Gene Disease-Related Site

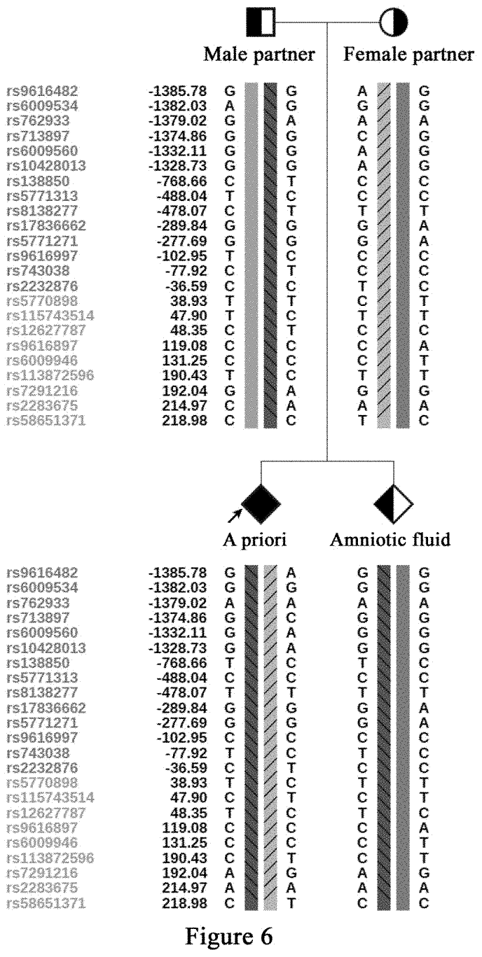

(1) Pedigree information: cardioencephalomyopathy due to cytochrome C oxidase deficiency, an autosomal recessive genetic disease with the pathogenic gene SCO2. The male partner was a carrier of SCO2 c.327_328del heterozygous mutation, and the female partner was a carrier of SCO2 c.551T>C heterozygous mutation. A born affected child carried a compound heterozygous mutation of SCO2 c.327_328del and c.551T>C. The female was pregnant, and amniotic fluid was taken to test the fetus for carrying the pathogenic site or not.
(2) The descendant object to be tested was amniotic fluid gDNA of the naturally conceived fetus. The gDNAs of the father and the mother (referred to as the male partner and the female partner hereinafter) of the descendant object were extracted. The reference object was the gDNA of another born child of the descendant object's parents.
(3) Polymorphic site genotype data were obtained by multiplex PCR and targeted second-generation sequencing of the gDNAs obtained from the male partner, the female partner, the affected child, and the fetal amniotic fluid.
(4) The subsequent analysis method was as described in Example 1.
(5) The analysis results were shown in Table 4 and FIG. 6.

TABLE 4
Summary of detection results in Example 4

| Sample | Aneuploidy detection | Analysis result of pathogenic site |
|---|---|---|
| name | result | and SNP linkage |
| Amniotic | 46, XN | Paternally carried |
| fluid | | |

### Example 5: A Born Child+Inference of the Carrying Status of Single-Gene Disease-Related Site

In Example 3, since both the male partner and the female partner carried the same heterozygous mutation, a child of the male partner and the female partner also carried the heterozygous mutation, it could not be determined whether the child carries the paternal mutation or the maternal mutation. Using the definite embryo phenotype results in Example 3, it could be inferred that the child carries the paternal mutation.

### Example 6: Determining a Haplotype of a Descendant Object

In this Example, the methods of Examples 1-4 were repeated with a difference that a reference data set C from a different reference object was used.

Particularly, the method was as follows:

(i) Providing s data sets for the analysis, wherein s is a positive integer greater than or equal to 4, wherein the data sets are related to genome information and comprise: a first data set derived from the descendant object, a second data set derived from the father of the descendant object, a third data set derived from the mother of the descendant object, and at least one reference data set C from a reference object;
wherein the reference object is a genetically related relative other than the father and the mother of the descendant object;
(ii) Selecting Y1 target sites, wherein Y1 is a positive integer greater than or equal to 1;
(iii) For each target site selected out in the previous step, analyzing and detecting molecular markers in the upstream and downstream regions of the target site, so as to determine at least one molecular marker upstream of and at least one molecular marker downstream of each target site;
(iv) Annotating each of the molecular markers determined in step (iii) in each of the data sets to obtain the corresponding first data set, second data set, third data set and reference data set C annotated with the molecular markers;
(v) For each of the molecular marker sites upstream and downstream of each target site in each of the data sets, constructing binary genetic vectors of (0, 1); n data sets constitute 2n vectors of Vᵢ, wherein i represents a site, and Vᵢ is a Hidden Markov Chain state; wherein n is s or s-j, and s is as defined above, and j is the number of the uppermost ancestral individuals without a parental generation (i.e., individuals without parents in the pedigree);
(vi) For each target site, determining a maximum likelihood estimation value L by Formula Q1 using a Hidden Markov model:

$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$

wherein:
m represents the number of molecular markers upstream and downstream of each target site;
P(Vᵢ) represents a priori value of a genetic vector;
P(Vᵢ|Vᵢ₋₁) represents a transition probability of the haplotype status between two adjacent sites;
Gᵢ represents an observed value of a genotype at the ith site;
P(Gᵢ|Vᵢ) represents an emission probability of a haplotype status; and
(vii) Estimating a maximum possible composition of V₁, V₂, . . . Vₘ by using a Viterbi dynamic programming algorithm, thus the haplotype of the descendant object is determined.

So far more than 100 clinical pedigree samples had been tested and verified. Several representative haplotype analysis results derived from using different reference objects were shown in FIGS. 7-12. These results showed that the method of the invention could accurately, flexibly and efficiently obtain haplotype analysis results.

### Example 7: Device for Analyzing a Haplotype of a Descendant Object

A device for analyzing a haplotype of a descendant object, comprising:

(a) A data input unit which is used for inputting s data sets for the analysis, wherein s is a positive integer greater than or equal to 4, wherein the data sets are related to genome information and comprise: a first data set derived from the descendant object, a second data set derived from the father of the descendant object, a third data set derived from the mother of the descendant object, and at least one reference data set C from a reference object;
(b) An analysis site annotation unit which is used for annotating analysis sites in each of the data sets, wherein the analysis sites are molecular markers identified by analysis and detection upstream and downstream regions of a predetermined target site;
(c) A haplotype analysis unit configured to perform the following operations:
(Y1) Determining a binary genetic vector of (0, 1) for each analysis site in each of the data sets;
(Y2) Determining a maximum likelihood estimation value L by Formula Q1 using a Hidden Markov model:

$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$

wherein:
m represents the number of molecular markers upstream and downstream of each target site;
P(V₁) represents a priori value of a genetic vector;
P(Vᵢ|Vᵢ₋₁) represents a transition probability of the haplotype status between two adjacent sites;
Gᵢ represents an observed value of a genotype at the ith site;
P(Gᵢ|Vᵢ) represents an emission probability of a haplotype status;
(Y3) Determining the haplotype (or the haplotype genetic flow) of the descendant object by estimating the maximum possible composition of V₁, V₂, . . . . Vₘ through a Viterbi dynamic programming algorithm; and
   (d) An output unit for outputting the analysis result of the haplotype analysis unit.

Additionally, the analysis device further comprises one or more units selected from the group consisting of:

(e) A sequencing unit for sequencing a nucleic acid sample to obtain genomic sequence data;
(f) A quality control unit for quality control of the molecular marker genotype data; and
(g) A genotype error processing unit for exclusion of genotype errors for a haplotype in each of the data sets.

### Discussion

At present, researchers have used haplotype linkage analysis strategies to detect the carrying status of pathogenic variations, such as the PGH (Preimplantation Genetic Haplotyping) technique using Microsatellite (Short Tandem Repeats, STR) markers (Renwick P. J., Trussler J., Ostad-Saffari E., Fassihi H., Black C., Braude P., Ogilvie C. M. and Abbs S. 2006, Proof of principle and first cases using preimplantation genetic haplotyping—a paradigm shift for embryo diagnosis, Reprod Biomed Online 13(1): 110-119) and the Karyomapping technique using Single Nucleotide Polymorphism (SNP) genotype (Handyside A. H., Harton G. L., Mariani B., Thornhill A. R., Affara N., Shaw M.A. and Griffin D, 2015, Karyomapping: a universal method for genome wide analysis of genetic disease based on mapping crossovers between parental haplotypes, J Assist Reprod Genet 32(3): 347-356). However, all these techniques share a common characteristic that, the haplotype inherited from the carrier parent is determined via one reference object (either a carrier of the pathogenic site or a normal individual) in the pedigree of the pathogenic site carrier. All other haplotypes are compared with the haplotype of the reference object, and the descendant object's status is inferred based on the carrying status of the pathogenic site in the reference object. Therefore, these methods have the following problems: \textcircled{1} Only one object is used for haplotype inference, and the accuracy of haplotype phasing is questionable; \textcircled{2} In cases where the reference object is not the siblings but, for example, the paternal grandparents, a maternal uncle, a maternal aunt, a younger paternal uncle or an elder paternal uncle, the informative sites which can be used for inferring the pathogenic state are limited, leading to decreased reliability of the inference. However, these cases are quite common in specific clinical practices, which brings some challenges to clinical applications. \textcircled{3} Different reference objects have different inference strategies, and different information sites available for inference, so it is not flexible.

The inventors of the present invention have developed a new haplotype analysis method and a device after long-term research. Particularly, the present invention provides a data analysis method for determining haplotype genetic flow; an analysis method for determining the haplotype of a descendant object; and a device for analyzing the haplotype of a descendant object.

Compared with the previous PGH and Karyomapping techniques that use only one reference object for linkage analysis, the method of the invention uses genetic information of all objects in the pedigree to carry out haplotype analysis. For example, the genotype information of a number of embryos can be used for mutual inference, thus contributing to a more accurate haplotype phasing.

In clinical applications, the more informative sites linkage analysis uses, the higher the inference accuracy gets. However, in specific clinical practice, whether it is targeted sequencing or genotyping chip detection, the sites for linkage analysis are limited, so the maximum utilization of existing sites is also one of the criteria for evaluating the methods. The method of the present invention can even use the genetic information of a number of embryos to infer the haplotype of the parental carriers of the embryos, and successfully achieve the haplotyping of a triple heterozygous site (for example, a heterozygous site carried by a parent and his/her parents of an embryo), thus there are more informative sites capable of being used to deal with cases where the reference object is not a sibling of the embryo, resulting in more reliable results.

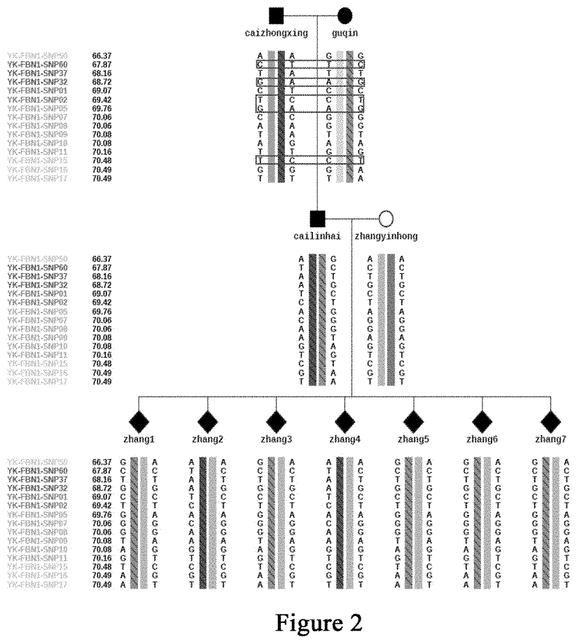

Taking FIG. 2 as an example, this is a case of an autosomal dominant genetic disease. The father of the embryos is a compound heterozygous mutation carrier. Using the method of the invention, the pathogenic site carrying status of the embryos are successfully inferred. In contrast, in the PGH and Karyomapping strategies, the paternal grandfather or the paternal grandmother of the embryos would be used as a reference object for the haplotyping of the embryos' father. Since the five sites in the red boxes in FIG. 2 are triple heterozygous sites (i.e., heterozygous sites for the father, the paternal grandfather and the paternal grandmother of the embryos), they cannot be used as informative sites for linkage analysis, such that there are not enough informative sites at the upstream of the pathogenic site for a reliable inference.

The method of the invention is convenient and flexible. The method can be used to analyze any type of reference objects, including siblings of an embryo, other family members, a single sperm, a polar body, etc. For single-gene genetic disorders, the method of the invention can be used to flexibly handle diseases with different inheritance patterns, such as autosomal dominant inheritance, autosomal recessive inheritance, X chromosome linked inheritance, etc.

On one hand, the method of the invention is particularly suitable for the situations of incomplete pedigree information, and on the other hand it can be used for applications in forensic identification such as parent-child identification.

All literatures mentioned in the invention are incorporated by reference in this application as if each literature is individually incorporated by reference. Furthermore, it should be understood that, various changes or modifications to the invention can be made by those skilled in the art after reading the above descriptions in the invention, and these equivalences also fall within the scope of the claims appended to this application.

What is claimed is:

1. A data analysis method for determining a haplotype genetic flow, characterized by comprising the following steps:
(a) Providing data sets for the analysis, wherein the data sets are related to genome information and comprise: a first data set derived from a descendant object, a second data set derived from the father of the descendant object and/or a third data set derived from the mother of the descendant object, and a reference data set C derived from at least one reference object; wherein the total number of the first, second, and third data sets and the reference data set C is s;
wherein the reference object is a genetically related relative other than the father and the mother of the descendant object; and
provided that:
(1) when both the second data set and the third data set are present, s is a positive integer greater than or equal to 4;
(2) when the second data set is present and the third data set is absent, s is a positive integer greater than or equal to 3, and the reference object is a genetically related relative other than the father and the mother of the descendant object and is genetically related to the father; and
(3) when the third data set is present and second data set is absent, s is a positive integer greater than or equal to 3, and the reference object is a genetically related relative other than the father and the mother of the descendant object and is genetically related to the mother;
(b) Performing molecular marker genotying in the upstream and downstream regions of Y1 target sites in each of the data sets, thereby obtaining molecular marker genotype data, wherein Y1 is a positive integer greater than or equal to 1;
(c) For each of the molecular marker sites upstream and downstream of each target site in each of the data sets, constructing binary genetic vectors of (0, 1); n data sets constitute 2n vectors of Vᵢ, wherein i represents a site, and Vᵢ is a Hidden Markov Chain state; wherein n is s or s-j, and s is as defined above, and j is the number of the uppermost ancestral individuals without a parental generation;
(d) For each target site, determining a maximum likelihood estimation value L by Formula Q1 using a Hidden Markov model:
$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$
wherein:
m represents the number of molecular markers upstream and downstream of each target site;
P(V₁) represents a priori value of a genetic vector;
P(Vᵢ|Vᵢ₋₁) represents a transition probability of the haplotype status between two adjacent sites;
Gᵢ represents an observed value of a genotype at the ith site;
P(Gᵢ|Vᵢ) represents an emission probability of a haplotype status; and
(e) Estimating a maximum possible composition of V₁, V₂, . . . Vₘ by using a Viterbi dynamic programming algorithm, thus a haplotype genetic flow direction of the descendant object and family members is determined.

2. An analysis method for determining a haplotype of a descendant object, characterized by comprising the following steps:
(i) Providing s data sets for the analysis, wherein s is a positive integer greater than or equal to 4, wherein the data sets are related to genome information and comprise: a first data set derived from the descendant object, a second data set derived from the father of the descendant object, a third data set derived from the mother of the descendant object, and at least one reference data set C from a reference object;
wherein the reference object is a genetically related relative other than the father and the mother of the descendant object;
(ii) Selecting Y1 target sites, wherein Y1 is a positive integer greater than or equal to 1;
(iii) For each target site selected out in the previous step, analyzing and detecting molecular markers in the upstream and downstream regions of the target site, so as to determine at least one molecular marker upstream of and at least one molecular marker downstream of each target site;
(iv) Annotating each of the molecular markers determined in step (iii) in each of the data sets to obtain the corresponding first data set, second data set, third data set and reference data set C annotated with the molecular markers;
(v) For each of the molecular marker sites upstream and downstream of each target site in each of the data sets, constructing binary genetic vectors of (0, 1); n data sets constitute 2n vectors of Vᵢ, wherein i represents a site, and Vᵢ is a Hidden Markov Chain state; wherein n is s or s-j, and s is as defined above, and j is the number of the uppermost ancestral individuals without a parental generation (i.e., individuals without parents in the pedigree);
(vi) For each target site, determining a maximum likelihood estimation value L by Formula Q1 using a Hidden Markov model:
$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$
wherein:
m represents the number of molecular markers upstream and downstream of each target site;
P(V₁) represents a priori value of a genetic vector;
P(Vᵢ|Vᵢ₋₁) represents a transition probability of the haplotype status between two adjacent sites;
Gᵢ represents an observed value of a genotype at the ith site;
P(Gᵢ|Vᵢ) represents an emission probability of a haplotype status; and
(vii) Estimating a maximum possible composition of V₁, V₂, . . . Vₘ by using a Viterbi dynamic programming algorithm, thus a haplotype of the descendant object is determined.

3. The method according to claim 1 or 2, characterized in that the P(Vᵢ|Vᵢ₋₁) is calculated by using a genetic map and obtaining a recombination rate; and/or
the P(Gᵢ|Vᵢ) is a probability calculated by combining an observed value of a sample genotype and a genotype of an ancestor thereof, and using the Mendelian inheritance law.

4. The method according to claim 1 or 2, characterized in that the descendant object is selected from the group consisting of humans or non-human mammals.

5. The method according to claim 1 or 2, characterized in that the method further comprises one or more features selected from the group consisting of:
(1) The data set is consisting of sequencing data or chip detection data of genome nucleic acids;
(2) The upstream and downstream regions comprise: ≤1 Mbp region, ≤2 Mbp region, ≤3 Mbp region or up to an entire chromosome;
(3) the molecular marker is selected from the group consisting of a SNP site, a STR polymorphic site, a RFLP site, an AFLP site, or a combination thereof;
(4) The molecular marker detection means include a microarray chip of single nucleotide polymorphic sites, a MassARRAY flight mass spectrometry chip, a MLPA multiplex ligation amplification technique, a second-generation sequencing, a third-generation sequencing, or a combination thereof;
(5) The molecular marker detection identifies for each target abnormal mutation at least two molecular markers that may be linked, and are recorded as analysis sites.

6. The method according to claim 2, characterized in that step (vii) further comprises: exclusion of a genotyping error site from within a haplotype.

7. The method according to claim 1 or 2, characterized in that the reference sample is selected from the group consisting of:
(Z1) an elder brother, a younger brother, an elder sister, or a younger sister of the descendant object (i.e., other descendants of the parents, including born and unborn), or a combination thereof;
(Z2) the father or mother of the father or mother of the descendant object, or a combination thereof;
(Z3) an elder brother, a younger brother, an elder sister, or a younger sister of the father or mother of the descendant object, or a combination thereof;
(Z4) an elder paternal uncle, a younger paternal uncle, a paternal aunt, a maternal uncle or a maternal aunt of the father or mother of the descendant object, or a combination thereof;
(Z5) the paternal grandfather, paternal grandmother, maternal grandfather or maternal grandmother of the father or mother of the descendant object, or a combination thereof;
(Z6) a sperm of the father of the descendant object, an ovum of the mother of the descendant object, a polar body (a first polar body or a second polar body) of the mother of the descendant object, or a combination thereof;
(Z7) any one of combinations of the Z1 to the Z6.

8. The method according to claim 1 or 2, characterized in that the estimating a maximum possible composition of V₁, V₂, . . . Vₘ is determining a maximum probability of the ancestral haplotype composition for each individual.

9. The method according to claim 2, characterized in that the method further comprises step (viii): visually displaying the abnormal mutation carrying status of the haplotype of the descendant object.

10. A device for analyzing a haplotype of a descendant object, characterized by comprising:
(a) A data input unit which is used for inputting s data sets for the analysis, wherein s is a positive integer greater than or equal to 4, wherein the data sets are related to genome information and comprise: a first data set derived from the descendant object, a second data set derived from the father of the descendant object, a third data set derived from the mother of the descendant object, and at least one reference data set C from a reference object;
(b) An analysis site annotation unit which is used for annotating analysis sites in each of the data sets, wherein the analysis sites are molecular markers identified by analysis and detection upstream and downstream regions of a predetermined target site;
(c) A haplotype analysis unit configured to perform the following operations:
(YT) Determining a binary genetic vector of (0, 1) for each analysis site in each of the data sets;
(Y2) Determining a maximum likelihood estimation value L by Formula Q1 using a Hidden Markov model:
$$L = {\sum }_{{V}_{1}} … {\sum }_{{V}_{m}} P ( {V}_{1} ) {\prod }_{i = 2}^{m} P ( {V}_{i} \vert {V}_{i - 1} ) {\prod }_{i = 1}^{m} P ( {G}_{i} \vert {V}_{i} ) \qquad ( Q1 )$$
wherein:
m represents the number of molecular markers upstream and downstream of each target site;
P(V₁) represents a priori value of a genetic vector;
P(Vᵢ|Vᵢ₋₁) represents a transition probability of the haplotype status between two adjacent sites;
Gᵢ represents an observed value of a genotype at the ith site;
P(Gᵢ|Vᵢ) represents an emission probability of a haplotype status;
(Y3) Determining the haplotype (or the haplotype genetic flow) of the descendant object by estimating the maximum possible composition of V₁, V₂, . . . Vₘ through a Viterbi dynamic programming algorithm; and
(d) An output unit for outputting the analysis result of the haplotype analysis unit.
